(19) United States
(12) Patent Application Publication
(10) Pub. No.: US 2014/0030727 A1
(43) Pub. Date: Jan. 30, 2014

(54) LOSS OF 5-HYDROXYMETHYLCYTOSINE AS A BIOMARKER FOR CANCER

(71) Applicants: Gerd Pfeifer, Duarte, CA (US); Seung-Gi Jin, Duarte, CA (US); Yong Jiang, Decatur, GA (US); Runxiang Qiu, Duarte, CA (US); Qiang Lu, Duarte, CA (US)

(72) Inventors: Gerd Pfeifer, Duarte, CA (US); Seung-Gi Jin, Duarte, CA (US); Yong Jiang, Decatur, GA (US); Runxiang Qiu, Duarte, CA (US); Qiang Lu, Duarte, CA (US)

(21) Appl. No.: 13/747,306

(22) Filed: Jan. 22, 2013

Related U.S. Application Data
(60) Provisional application No. 61/589,231, filed on Jan. 20, 2012.

(51) Int. Cl. G01N 33/574; G01N 33/483

(52) U.S. Cl. CPC G01N 33/57496; G01N 33/4833
USPC 435/6.14

(57) ABSTRACT

Methods for detecting or diagnosing cancer in a subject are provided herein. Such methods may include, but are not limited to, measuring a test level of 5hmC in a biological sample from the subject; and determining that the subject has a malignant cancer when the test level of 5hmC is lower than that of a control level of 5hmC. Such methods may further include a step of measuring a test level of Ki67 in the biological sample and determining that the subject has a malignant cancer when the test level of Ki67 is higher than that of a control level of Ki67.

### CROSS-REFERENCE TO RELATED APPLICATIONS

This application claims the benefit of U.S. Provisional Patent Application No. 61/589,231, filed Jan. 20, 2012, which is incorporated herein by reference in its entirety.

### GOVERNMENT SUPPORT

The present invention was made with government support under Grant Nos. CA084469, AG036041, NS075393, and CA101864, awarded by the National Institutes of Health. The Government has certain rights in the invention.

### BACKGROUND

5-hydroxymethylcytosine (5hmC) is a DNA pyrimidine nitrogen base that is formed from cytosine by adding a methyl group and a hydroxyl group. 5hmC is an oxidation product of 5-methylcytosine (5mC) in mammalian DNA (Kriaucionis & Heintz 2009; Tahiliani et al. 2009) mediated by the TET family of genes that encode, for example, alpha-ketoglutarate-dependent Tet dioxygenases. Levels of 5hmC are tissue dependent and the highest levels have been found in the central nervous system (Globisch et al. 2010; Szwagierczak et al. 2010). The distribution of 5hmC in embryonic stem cells (Ficz et al. 2011; Koh et al. 2011; Pastor et al. 2011; Wu et al. 2011; Williams et al. 2011; Xu et al. 2011b), mouse cerebellum (Song et al. 2011), and human prefrontal cortex (Jin et al. 2011) has been mapped by array- or sequencing-based assays. The specific biological function of 5hmC is poorly understood, but it may be involved with regulating gene expression or the process of DNA methylation. This is supported by data suggesting that 5hmC may be an intermediate in DNA demethylation processes that accomplishes the conversion of 5mC to cytosine (Wu & Zhang 2010) and that 5hmC is enriched at promoters and within gene bodies.

Several hematologic malignancies carry mutations in one of the TET genes, TET2 (Delhommeau et al. 2009). TET2 mutations have been linked to aberrant levels of 5hmC and 5mC in these cancer genomes (Ko et al. 2010; Figueroa et al. 2010). Moreover, mutations in isocitrate dehydrogenase-1 (IDH1) have been linked to abnormal DNA methylation patterns (Noushmehr et al. 2010). Therefore, it has been suggested that mutated IDH1 produces a new metabolite, 2-hydroxyglutarate (2HG; Dang et al. 2009), which can inhibit TET proteins resulting in altered levels of 5hmC and 5mC in tumors (Xu et al. 2011b). Because systematic studies on levels of 5hmC in human cancers are lacking, it would be desirable to determine whether 5hmC may be used as a cancer biomarker.

### SUMMARY

In one embodiment, a method for detecting or diagnosing cancer in a subject may include, but is not limited to, measuring a test level of 5hmC in a biological sample from the subject; and determining that the subject has a malignant cancer when the test level of 5hmC is lower than that of a control level of 5hmC. Such a method may further include a step of measuring a test level of Ki67 in the biological sample and determining that the subject has a malignant cancer when the test level of Ki67 is higher than that of a control level of Ki67.

In other embodiments, a method for detecting or diagnosing cancer in a subject may include, but is not limited to, measuring a test level of 5hmc in a biological sample from the subject; measuring a test level of Ki67 in the biological sample; and determining that the subject has a malignant cancer when (1) the test level of 5hmc is lower than that of a control level of 5hmc and (2) the test level of Ki67 is higher than that of a control level of Ki67.

In some embodiments, the biological sample is a tumor tissue sample and the test level of 5hmC may be measured using immunohistochemistry (1HC). In other embodiments, the biological sample is a DNA sample isolated from a tumor tissue and the test level of 5hmC may be measured using liquid chromatography/tandem mass spectrometry (LC/MS-MS). In such case, the test level of 5hmC may be measured as 5hmdC (5-hydroxymethyldeoxycytidine). The control level of 5hmC may be measured using a normal adjacent tissue from the same subject, a normal sample from a second subject or a population of normal subjects.

The methods described herein may be used to detect or diagnose any type of cancer including, but not limited to, bone cancer, bladder cancer, brain cancer, breast cancer, cancer of the urinary tract, carcinoma, adenocarcinoma, cervical cancer, colon cancer, esophageal cancer, gastric cancer, head and neck cancer, hepatocellular cancer, liver cancer, lung cancer, lymphoma and leukemia, malignant mesenchymoma, melanoma, neuroblastoma, ovarian cancer, pancreatic cancer, pituitary cancer, prostate cancer, rectal cancer, renal cancer, rhabdomyosarcoma, sarcoma, testicular cancer, thyroid cancer, or uterine cancer.

### BRIEF DESCRIPTION OF THE DRAWINGS

FIG. 1 shows an analysis of 5hmdC by LC-MS/MS. (A) shows the reaction pathway from cytosine (C) to 5-methylcytosine (5mC) and 5-hydroxymethylcytosine (5hmC). (B) shows typical selected-ion chromatograms (SIGs) for monitoring 5hmdC (a), 5mdC (c) and dG (d) derived from a digested DNA sample of normal human brain DNA mixed with the two stable isotope labeled standards of 5hmdC (b) and dG (e). Shown in the inserts are chemical structures for each deoxynucleoside and corresponding selected reaction monitoring (SRM) transition in the triple stage quadrupole (TSQ) mass spectrometer; the stable isotope labels are indicated by arrows.

FIG. 2 shows mass calibration curves for normal 5hmdC vs. labeled 5hmdC (a), normal dG vs. labeled dG (b) and normal 5mdC vs. labeled dG (c) measured by the TSQ mass spectrometer. An 897-bp DNA standard containing 100% 5hmdC (Zymo Research; Irvine, Calif.) was used to generate the mass Calibration curves for labeled and corresponding unlabeled nucleosides of 5hmdC and dG. The same amount of labeled 5hmdC and dG standards as used in sample analysis were mixed with different concentrations of digested DNA standard. The peak area ratios of 5mdC to labeled dG were calibrated to the actual ratios of deoxynucleosides in the standard mixture. In addition, an 897-bp DNA standard containing 100% 5-mdC (Zymo Research) was used to construct the calibration curve of 5mdC using labeled dG as the standard. The peak area ratios of 5mdC to labeled dG were calibrated to the actual ratios of deoxynucleosides in the standards mixture. The ratios of unlabeled versus labeled 2 standards were chosen to be in the expected range of the sample deoxynucleoside. Linear equations were used for calculation of the precise 5hmdC, 5mdC and dG quantities in the samples; 5hmdC vs. dG and 5mdC vs. dG were used to present 5hmdC and 5mdC levels of the samples. dG was chosen as a baseline standard, because it pairs with all three 2′-deoxycytidine derivatives: dC, 5mdC and 5hmdC. All calibration curves were constructed with three independent measurements.

FIG. 3 shows results of a quantitation analysis of 5hmdC in various mouse and human tissues and cell types. In (A), 5hmdC was measured in DNA from mouse sperm, mouse embryonic stem cells, mouse brain, human brain, the 293T cell line, a small cell lung cancer cell line, and in human fibroblasts. Measurement of 5mdC in the same samples is shown in (B). All assays were done in triplicate.

FIG. 4 shows an immuno dot blot analysis of 5hmC in normal brain and brain tumors. Normal brain DNA from prefrontal cortex (BN samples) and DNA from brain tumors (BT samples) (200 ng each) were spotted onto nylon membranes, which were probed with anti-5hmC (A) or anti-5mC (B) antibody as described in Jin et al. (2011) Nucleic Acids Res 39: 5015-5024. The samples were analyzed by LC-MS/MS (see FIG. 7).

FIG. 5 shows results of a quantitation analysis of 5hmdC and 5mdC in normal lung and squamous cell carcinoma (SCC) DNA. A shows quantitation results for 5hmdC. The first 18 samples are matched normal lung (LN, blue) and lung tumors (LT, green). The last 6 samples are lung tumors without available normal tissue. B shows quantitation results for 5mdC. The asterisks (*) indicate that the levels of 5mdC were significantly reduced in the tumor when compared with normal lung (P<0.05).

FIG. 6 shows results of a quantitation analysis of 5hmdC and 5mdC in lung small cell carcinomas and adenocarcinomas. A shows quantitation results for 5hmdC levels in primary small cell lung cancer. N, normal lung; T, small cell lung cancer. B shows quantitation results for 5mdC levels in primary small cell lung cancer. C shows quantitation results for 5hmdC levels in lung adenocarcinomas. LN, normal lung; LT, lung tumor. D shows quantitation results for 5mdC levels in lung adenocarcinomas. The analysis was performed by LC-MS/MS as described in the Materials and Methods.

FIG. 7 shows results of a quantitation analysis of 5hmdC and 5mdC in normal brain DNA and in stage II/III astrocytomas. (A) shows results for 5hmdC quantitation in normal brain (NB, (1)) and in brain tumors (BT, (2) or (3)). Samples BT1-16, BT25, BT27-29, and BT32-36 were stage III astrocytomas; BT17-24, BT26, BT30, and BT31 were stage II astrocytomas; and BT37 and BT38 were glioblastomas. (B) shows quantitation results for 5mdC in NB and in BTs. Tumors with no IDH1 mutation are indicated by (2); tumors with IDH1 R132H are indicated by (3). The sample BT26 had a minor allele frequency of IDH1 R132H. Sample BT25 had the rare mutation R132G.

FIG. 8 shows results of a quantitation analysis of 5hmdC and 5mdC in neurons and astrocytes of human fetal brain. (A) shows results for 5hmdC and (B) shows results for 5mdC. The analysis was performed by LC-MS/MS as described in the Materials and Methods.

FIG. 9 shows IDH1 and IDH2 mutations in brain and lung tumor samples. A shows sequencing scans that show a wildtype sequence, the common IDH1 R132H mutation, a minor allele fraction of R132H in tumor BT26 and a mutation IDH1 R132G in tumor BT25. B shows a summary of the mutation status of IDH1/2 in lung and brain tumors. Mutations of IDH2 in brain tumors are extremely rare and were not determined (N.D.).

FIG. 10 is a schematic model for inhibition of TET oxidase activity and 5hmC production by the oncometabolite 2-hydroxyglutarate (2HG) produced by mutant IDH1.

FIG. 11 shows an immunohistochemical analysis of 5hmC on human tissue arrays. Human tissue arrays containing samples of malignant tumor and corresponding normal tissue were stained with anti-5hmC antibody. Staining with Hoechst 33258 is shown as a control. The magnification of all panels is 10-fold.

FIG. 12 shows an immunohistochemical analysis of 5hmC on additional human tissue arrays. Human tissue arrays containing paired samples of malignant tumor and corresponding normal tissue (breast, colon, skeletal muscle, stomach, prostate, and ovary) were stained with anti-5hmC antibody and detected with secondary antibody conjugated to Rhod Red-X-AffiniPure. The magnification of all panels is 10-fold. Hoechst 33258 staining (blue) is shown as a control.

FIG. 13 shows analysis of additional lung tumors. A shows 5hmC staining of additional lung squamous cell (SCC) and adenocarcinomas. Results are shown for matched pairs of tumor-adjacent normal tissue and two squamous cell carcinomas (sample 509019, panels a and b; sample B509018, panels c, d, and e) and two adenocarcinomas (sample B509016, panels f and g; sample B509015; panels h, i, and j). Boundary sections between normal and tumor are also shown (panels d and i). B shows a comparison of 5hmC and Ki67 staining in normal lung and in SCC lung tumors. The staining for 5hmC and Ki67 is mutually exclusive. Red, 5hmC; green, Ki67. All Ki67-positive cells lack 5hmC staining.

FIG. 14 shows co-staining with anti-5hmC and anti-5mC antibodies. A shows a normal brain section and a brain tumor; B shows a normal liver and a liver tumor; and C shows a normal kidney and a kidney tumor.

FIG. 15 shows staining of 5hmC and Ki67 antigen in brain and brain tumors. (A) shows that Ki67 staining of normal brain sections and brain tumors shows absence of Ki67 staining in normal brain. (B) shows mostly mutually exclusive staining for Ki67 and 5hmC in brain tumors. Data for two brain tumors are shown.

FIG. 16 illustrates an inverse relationship between 5hmC and Ki67 staining. Sections of normal lung and lung tumor, normal prostate and prostate tumor, and normal small intestine were stained with anti-5hmC (indicated by dull grey staining) and anti-Ki67 antibodies (indicated by bright white staining) to mark proliferating cells. Note the mutually exclusive staining of 5hmC and Ki67 in the tissue sections. For example, proliferating cells in intestinal crypts are positive for Ki67 but negative for 5hmC.

FIG. 17 shows an analysis of 5hmC and Ki67 in uterus, breast and pancreas tissue and tumors. Dull grey staining indicates 5hmC positive cells; bright white staining indicates Ki67 positive cells. Ki67-positive cells lack 5hmC staining. Note that not all cells that lack 5hmC staining in the tumors are Ki67-positive presumably due to past history of proliferation leading to persistent loss of 5hmC.

FIG. 18 shows expression of the TET1, TET2, and TET3 genes in normal brain and astrocytic gliomas and in normal lung and lung squamous cell carcinomas. A. Brain. BN, normal brain; BT, brain tumor. B. Lung. LN, normal lung; LT, lung tumor. Gene-specific RT-PCR data for all three TET genes were normalized to beta-actin levels. PCR was performed with the TaqMan MGB primer with 6FAM-based probes (Applied Biosystems) using the following assay ID numbers: TET1 (Hs00286756_m1), TET2 (Hs00325999_m1), TET3 (Hs00379125_m1). Although expression levels of certain TET genes were higher in some tumors than in corresponding normal tissue, no generalized trend was observed. Overall, TET expression levels were not reduced in tumors, and there was no correlation between TET expression and levels of 5hmdC (FIGS. 5 and 7).

### DETAILED DESCRIPTION

Methods for detecting or diagnosing cancer in a subject, determining whether a subject has a malignancy or determining whether a tumor is malignant by measuring levels 5-hydroxymethylcytosine (5hmC) are provided herein. The base 5-hydroxymethylcytosine (5hmC) has been identified as an oxidation product of 5-methylcytosine in mammalian DNA. As described further below, sensitive and quantitative methods were used to assess levels of 5-hydroxymethyl-2′-deoxycytidine (5hmdC) and 5-methyl-2′-deoxycytidine (5mdC) in genomic DNA to show that the level of 5hmC can distinguish normal tissue from tumor and cancerous tissue. Because 5hmc is able to distinguish between normal, tumor and cancerous tissue, it may also serve as a target for treatment, monitoring and prognosis of subjects diagnosed with cancer resulting from the methods described herein.

According to the embodiments described herein, the methods for detecting or diagnosing cancer in a subject, determining whether a subject has a malignancy or determining whether a tumor is malignant described herein may include, but are not limited to, a step of detecting a loss or a lack of 5hmC in a biological sample from the subject. A diagnosis may refer to the detection, determination, or recognition of a health status or condition of a subject. In certain embodiments, the diagnostic method may detect, determine, or recognize the presence or absence of a cancer; a specific stage, type or sub-type, or other classification or characteristic of the cancer; whether a tumor is a benign lesion or a malignant tumor, or a combination thereof. In other embodiments, the methods described herein may also be used to differentiate between an early stage (i.e., an AJCC stage I-II cancer) or locoregional cancer (i.e., an AJCC stage I-III cancer); and a late stage (i.e., an AJCC stage III-IV cancer) or a cancer that has progressed to a cancer with visceral or distant metastasis (i.e., an AJCC stage IV cancer) when the test level is significantly different than the control level.

Detecting a loss or a lack of 5hmC in a biological sample may include, but is not limited to, measuring a test level of 5hmC in a biological sample and determining that the subject has cancer when the test level of 5hmC is lower than that of a control level of 5hmc. A test level, expression level or other calculated test level of a nucleotide, nucleoside, nucleic acid, nucleic acid transcript, peptide or protein, modification thereof, or other biomarker refers to an amount of a biomarker (e.g., 5hmC, Ki67) in a subject's undiagnosed biological sample. The test level may be compared to that of a control sample, or may be analyzed based on a reference standard that has been previously established to determine a status of the sample. A test level or test amount can be measured in an absolute amount (e.g., percentage of an internal or external standard, ratio of number of copies/mL, nanogram/mL or microgram/mL) or a relative amount (e.g., relative intensity of signals).

A control level, expression level or other calculated level of a nucleotide, nucleoside, nucleic acid, nucleic acid transcript, peptide or protein, modification thereof, or other biomarker may be any amount or a range of amounts to be compared against a test amount of a biomarker. A control level may be the amount of a marker in a healthy or non-diseased state. For example, a control amount of a marker can be the amount of a marker in a population of patients with a specified condition or disease or a control population of individuals without said condition or disease. A control amount can be either an absolute amount (e.g., number of copies/mL, nanogram/mL or microgram/mL) or a relative amount (e.g., relative intensity of signals). Alternatively, a control amount may include a range.

The test level or control level of 5hmC, or 5mC used in the methods described herein may be measured, quantified and/or detected by any suitable detection, quantification or sequencing methods known in the art. In one embodiment, a level of 5mC, 5hmC or both may be detected or measured using liquid chromatography/tandem mass spectrometry (LC/MS-MS). When LC-MS-MS is used, the DNA is treated with one or more enzymes to generate single nucleosides for quantitative analysis, therefore, the level of 5mC and 5hmC are measured as a level of 5mdC, and 5hmdC, respectively. As discussed in the Examples below, liquid chromatography/tandem mass spectrometry (LC/MS-MS) was used to assess the levels of 5-hydroxymethyl-2′-deoxycytidine (5hmdC) and 5-methyl-2′-deoxycytidine (5mdC) in human lung carcinomas and in brain tumor DNA. In some embodiments, a test level or test amount of 5hmdC may be expressed a percentage of dG, as described below. Test levels of 5hmdC in squamous cell lung cancers were substantially depleted with up to a 5-fold reduction as compared to normal lung tissue (control). In brain tumors, 5hmdC showed an even more drastic reduction with levels up to and more than 30-fold lower than in normal brain, but 5hmdC levels were independent of mutations in isocitrate dehydrogenase-1 (IDH-1). Thus, in some embodiments, a subject may be determined to have malignant cancer when the test level of 5hmdC is between approximately 2 to 5-fold lower than the control; approximately 2-fold lower than the control, approximately 3-fold lower than the control, approximately 4-fold lower than the control, approximately 5-fold lower than the control, approximately 10-fold lower than the control, approximately 20-fold lower than the control, approximately 30-fold lower than the control, or more than 30-fold lower than the control.

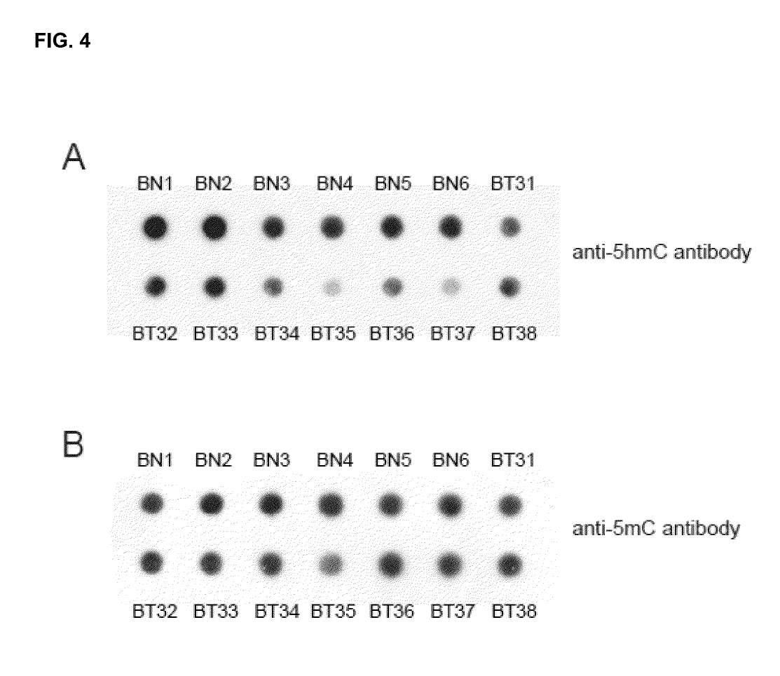

In another embodiment, levels of 5hmC may be detected by immuno dot blot (see FIG. 4), wherein DNA from control and test samples is spotted onto a nylon membrane. The membrane is incubated with an antibody against 5hmC. The binding of the antibody is detected by chemiluminescence, in a same or similar approach as in Western blotting).

In another embodiment, a level of 5mC, 5hmC or both, may be detected or measured using an immunofluorescence method, IHC method or other method that utilizes an antibody to detect the level of 5mC, 5hmC or both in a tissue. As described below, immunofluorescence staining was used to assess 5hmC in a series of normal and malignant tissue sections. Furthermore, immunohistochemical analysis indicated that 5hmC is significantly depleted in many types of human cancer. Importantly, an inverse relationship between 5hmC levels and cell proliferation was observed, wherein a lack of 5hmC was also observed in proliferating cells stained with Ki67. The data therefore suggest that 5hmdC is strongly depleted in human malignant tumors, a finding that adds another layer of complexity to the aberrant epigenome found in cancer tissue. In addition, a lack of 5hmC may be used as a biomarker for cancer diagnosis.

Therefore, according to some embodiments, methods for diagnosing cancer may include detecting a lack or loss of 5hmC alone as described herein or in combination with the use of additional biomarkers for detecting cell proliferation. For example, detection of a lack or loss of 5hmC may be performed in combination with a diagnostic immunochemical test for cancer that includes staining a biological sample using an antibody that targets proliferating cells including, but not limited to, an anti-Ki67 antibody, an anti-PCNA antibody, an anti-cyclin D1 antibody, an anti-cyclin E1 antibody, an anti-cyclin B1 antibody, an anti-MCM gene antibody, an anti-E2F1 antibody or an anti-PLK1 antibody. In one embodiment, such a method includes steps of measuring a test level of Ki67 in a biological sample and determining that a subject has a malignant cancer when the test level of Ki67 is higher than that of a control level of Ki67. Measuring a test level of Ki67 in combination with measuring a test level of 5hmC (as described above) offers an improved diagnostic tool as compared to testing with Ki67 staining alone because not all tumors contain large numbers of Ki67-positive cells and may be missed. As shown in the Examples herein, Ki67-positive cells generally lack 5hmC. However other cells in the tumor, which may be Ki67-negative also lack 5hmC suggesting that these cells are currently dormant but had a prior history of proliferation that led to loss of 5hmC.

Cancers and tumor types that may be detected or diagnosed using the methods described herein include but are not limited to bone cancer, bladder cancer, brain cancer, breast cancer, cancer of the urinary tract, carcinoma, adenocarcinoma, cervical cancer, colon cancer, esophageal cancer, gastric cancer, head and neck cancer, hepatocellular cancer, liver cancer, lung cancer, lymphoma and leukemia, malignant mesenchymoma, melanoma, neuroblastoma, ovarian cancer, pancreatic cancer, pituitary cancer, prostate cancer, rectal cancer, renal cancer, rhabdomyosarcoma, sarcoma, testicular cancer, thyroid cancer, and uterine cancer. In addition, the methods may be used to diagnose tumors that are malignant (e.g., primary or metastatic cancers) or benign (e.g., hyperplasia, cyst, pseudocyst, hematoma, and benign neoplasm).

As described herein, a biological sample refers to any material, biological fluid, tissue, or cell obtained or otherwise derived from a subject that contains or may contain DNA including, but not limited to, blood, plasma, serum, sputum, tears, mucus, nasal washes, nasal aspirate, breath, urine, semen, saliva, meningeal fluid, amniotic fluid, glandular fluid, lymph fluid, milk, bronchial aspirate, synovial fluid, joint aspirate, tumor tissue (e.g., a malignant tumor, a benign tumor or an unknown tumor tissue type), healthy tissue, cells, a cellular extract, and cerebrospinal fluid. A biological sample may also include materials containing homogenized solid material, such as from a stool sample, a tissue sample, or a tissue biopsy; or materials derived from a tissue culture or a cell culture. If desired, a sample may be a combination of samples from an individual, such as a combination of a tissue and fluid sample.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1

### 5-hydroxymethylcytosine is strongly depleted in human cancers but its levels do not correlate with IDH1 mutations

### Materials and Methods

DNA Samples.

Stage-I lung squamous cell carcinoma (SCC) and adenocarcinoma samples and matched normal tissues were obtained from the frozen tumor bank of the City of Hope Medical Center under an Institutional Review Board approved protocol. Samples were obtained from tumors without laser-capture micro-dissection. DNA from primary small cell lung cancers and matched normal lung was obtained from Asterand, BioChain, and Cureline. Normal human brain tissue DNA samples of the pre-frontal cortex were obtained from Capital Biosciences and BioChain. DNA from neurons and astrocytes of fetal (24 weeks of gestation) human brain was obtained from ScienCell. Twenty-seven astrocytomas (World Health Organization, grade II-III) were obtained on Institutional Review Board approved protocols at the Department of Neurosurgery at the University Hospital in Dresden. DNA was isolated by standard procedures with phenol-chloroform extraction and ethanol precipitation. Eight additional brain tumor DNA samples were obtained from Asterand. Genomic DNA samples from tissues and cell lines were isolated using a DNeasy Tissue Kit (QIAGEN).

IDH Mutations.

For sequencing of IDH1 and IDH2 exon 4, 40 ng of genomic DNA was used for PCR amplification using the following primers: for IDH1, forward 50-TGCCACCAACGACCAAGTCA (SEQ ID NO:1) and reverse 50-CATGCAAAATCACATATTTGCC (SEQ ID NO:2); for IDH2, forward 50-TGAAAGATGGCGGCTGCAGT (SEQ ID NO:3) and reverse 50-GGGGTGAAGACCATTTTGAA (SEQ ID NO:4).

Simultaneous Quantification of 5mdC and 5hmdC by LC/MS-MS.

Genomic DNA (1-2 μg) was incubated with 5 units of DNA Degradase Plus (Zymo Research) at 37° C. for at least 2 hours. The stable isotope labeled 5hmdC (Cao & Wang 2006; LaFrancois et al. 1998) and labeled 2′-deoxyguanosine (Cambridge Isotope Laboratories) were added as internal standards. Aliquots of the mixture were subjected directly to LC/MS-MS analysis. LC/MS-MS was carried out with a Thermo Accela 600 HPLC pump interfaced with a TSQ Vantage triple stage quadruple mass spectrometer (Thermo Fisher Scientific). A 2.1×50 mm Kinetex XB-C18 column (2.6 μm in particle size and 100 Å in pore size; Phenomenex) was used for separation at a flow rate of 400 mL/min. The TSQ mass spectrometer was optimized and set up in selected reaction monitoring scan mode for monitoring the [M+H]⁺ ions of 5hmdC (m/z 258.1→142.1), 5mdC (m/z 242.1→126.1), dG (m/z 268.1→152.1), labeled 5hmdC (m/z 261.1→144.1) and labeled dG (m/z 273.1→157.1). Thermo Xcalibur software (version 2.1) was used to conduct data analysis. Immunodot blot analysis for 5hmC was conducted as described previously (Jin et al. 2011).

Immunohistochemistry.

Frozen tissue arrays were from Biochain (catalog no. T6235700-5 and lot no. B403109). They contain normal brain tissue and craniopharyngioma, normal breast and invasive ductal carcinoma, normal colon and adenocarcinoma, normal skeletal muscle and rhabdomyosarcoma, normal kidney and renal cell carcinoma, normal liver and hepatocellular carcinoma, normal lung and SCC, normal pancreas and adenocarcinoma, normal prostate and adenocarcinoma, normal skin and malignant melanoma, normal small intestine and malignant mesenchymoma, normal stomach and adenocarcinoma, normal uterus and adenocarcinoma, and normal ovary and cystadenocarcinoma. The tissue sections were boiled in 10 mmol/L sodium citrate for antigen retrieval followed by blocking with 10% goat serum, 0.1% Triton X-100 in PBS for 1 hour at room temperature (RT). Sections were incubated with primary anti-5hmC polyclonal antibody (dilution 1:1,000; ActiveMotif) in 5% goat serum, 0.01% Triton X-100 in PBS at 4° C., overnight. After washing with PBS at RT, sections were incubated with Rhod Red-X-AffiniPure conjugated goat anti-rabbit secondary antibody (dilution 1:200; Jackson ImmunoResearch) for 1 hour at RT, then washed with PBS and water, and mounted with fluoromount-G solution (SouthernBiotech).

Ki67 staining was carried out with a Ki67 antibody (BD Pharmingen; catalog number 550609; dilution 1:20). The anti-5mC antibody was from Eurogentec (catalog no. BI-MECY-0100; dilution 1:200). Slides were counterstained with Hoechst 33258 dye. All fluorescent images were taken using an inverted Olympus IX 81 fluorescence microscope.

Reverse Transcriptase PCR.

Quantitative reverse transcriptase PCR was carried out as previously described (Iqbal et al. 2011).

Results and Discussion

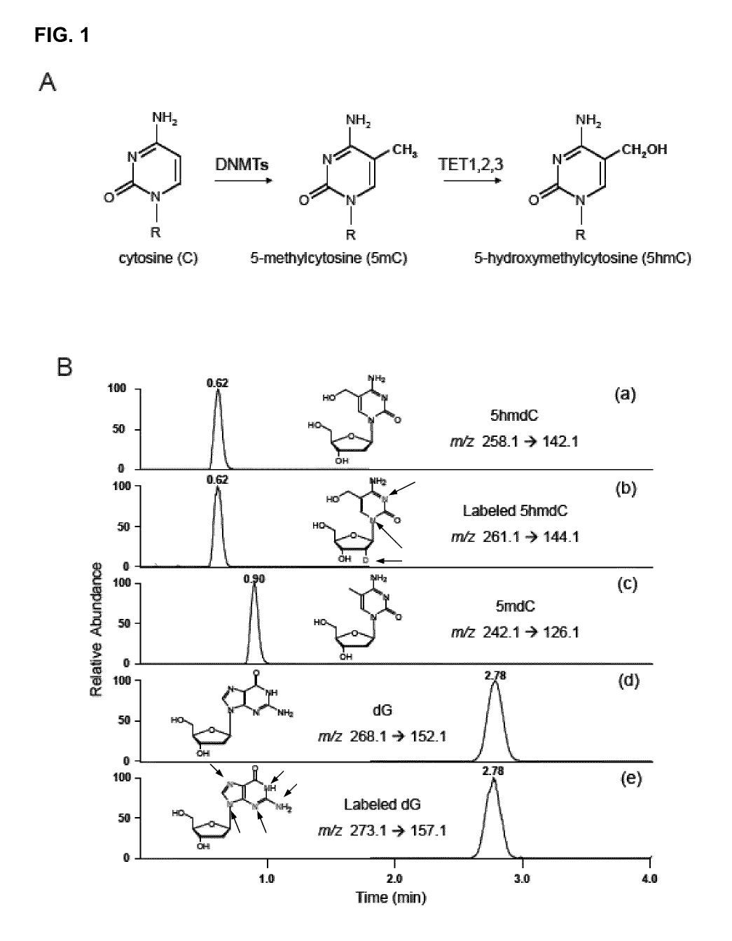

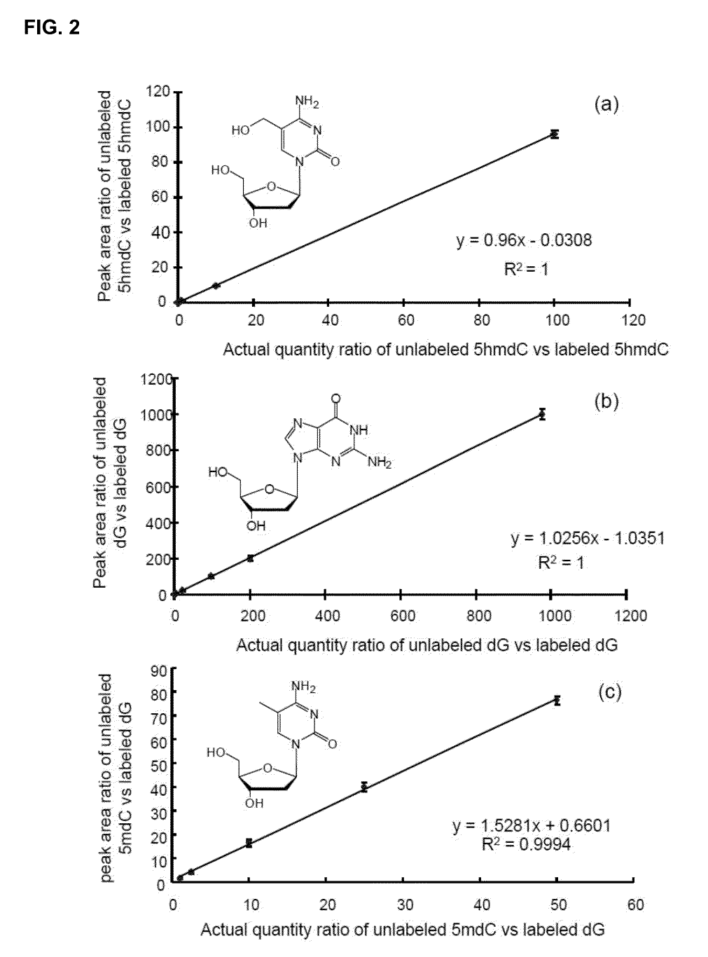

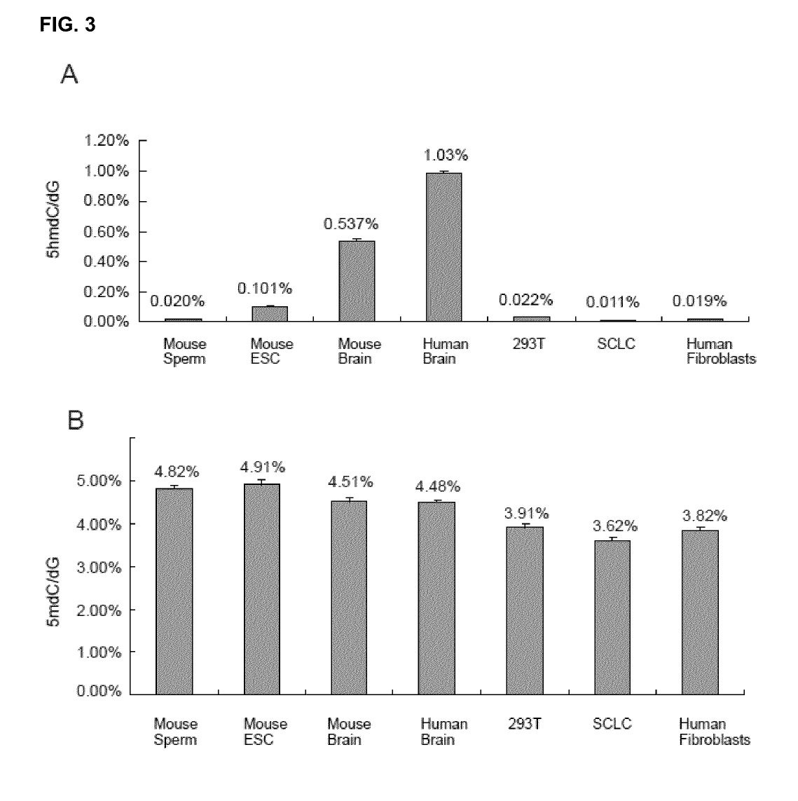

To determine the levels of 5hmdC and 5mdC in normal and tumor tissues, a sensitive LC/MS-MS assay was developed with isotope-labeled internal standards (FIG. 1A). 5mdC was quantitated with reference to the dG standard. FIG. 1B shows examples of LC separation and how mass spectrometric analysis of 5hmdC and 5mdC was achieved. The method is strictly quantitative as shown by standard curves (FIG. 2) and its performance was initially tested by measuring 5hmdC and 5mdC in several cell and tissue DNA samples (FIG. 3). The data obtained were consistent with values reported in the literature (Tahiliani et al. 2009; Globisch et al. 2010) and were also generally in agreement with a less quantitative immunodot blot assay (FIG. 4).

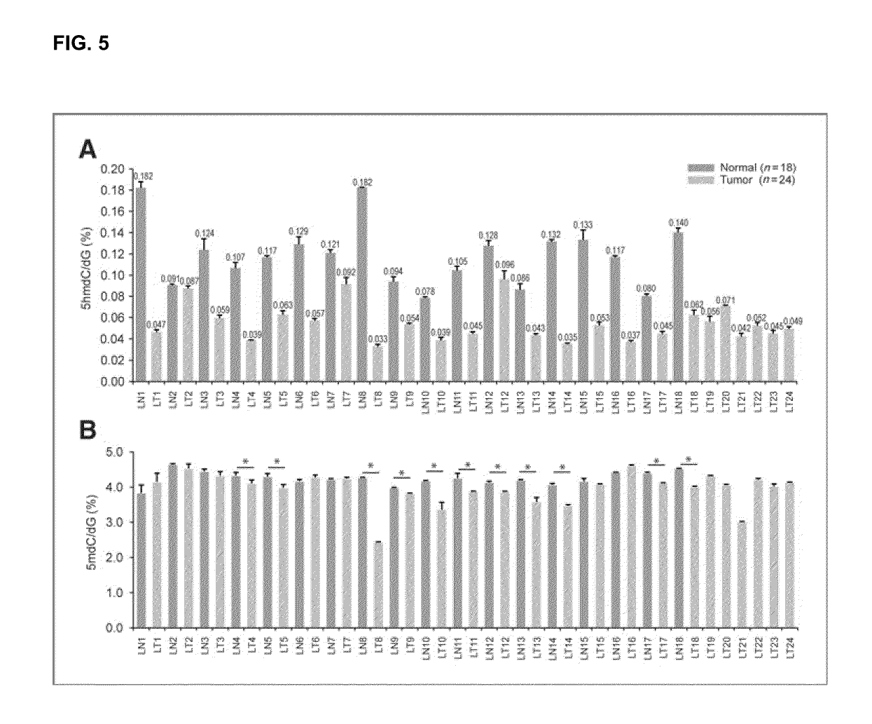

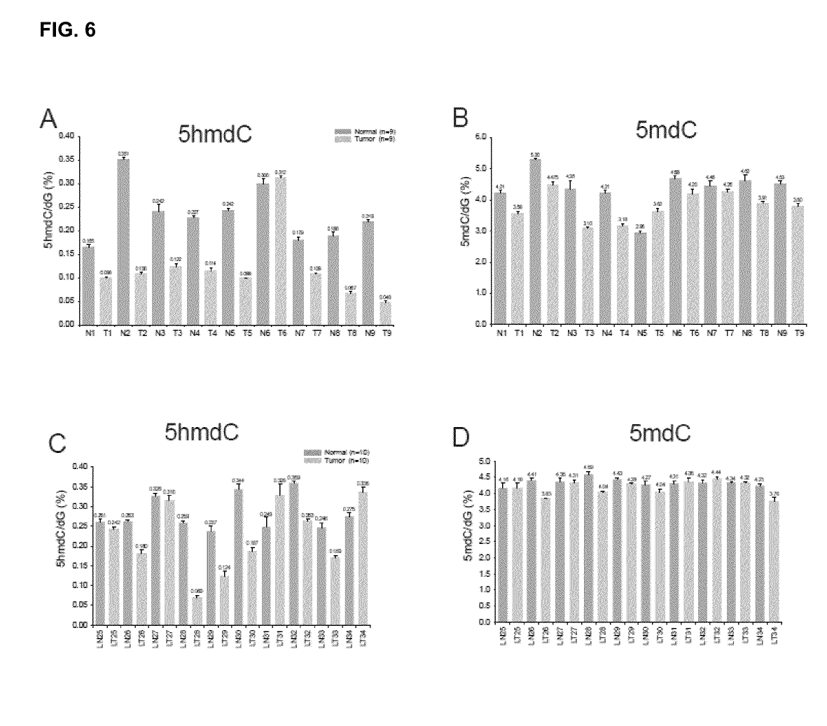

Using the LC/MS-MS assay, 5hmdC was measured in 24 stage-I lung SCC DNA samples and in matched normal lung DNA (FIG. 5A). The levels of 5hmdC, expressed as percentage of dG, were between 0.078% and 0.182% in normal lung. In every SCC tumor except one (LT2), a significant reduction of 5hmdC level as compared to the paired normal lung sample was observed (P<0.05 for each sample pair; t test; FIG. 5A). 5hmdC levels were generally 2- to 5-fold lower in the tumors than in normal lung (P=8.88×10⁻⁷; paired t test). 5mdC (FIG. 5B) was also quantitated. 5mdC was depleted in most tumor samples with a few exceptions (tumors 1, 2, 6, 7, 15, and 16). In many cases, 5mdC levels were lower by only approximately 5% to 20% (P=0.023; paired t test). IDH1 or IDH2 mutations were not found in these lung tumors. 5hmdC was also analyzed in lung adenocarcinomas and primary small cell lung cancers (FIG. 6). As with SCC, 5hmdC was depleted in most of these tumors relative to matched normal tissue.

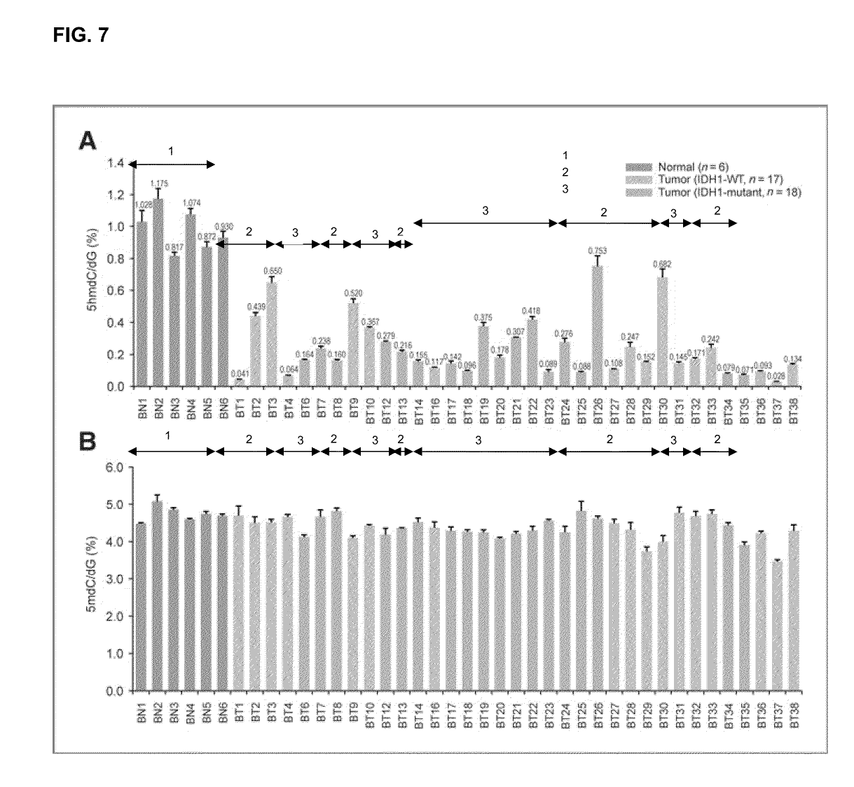

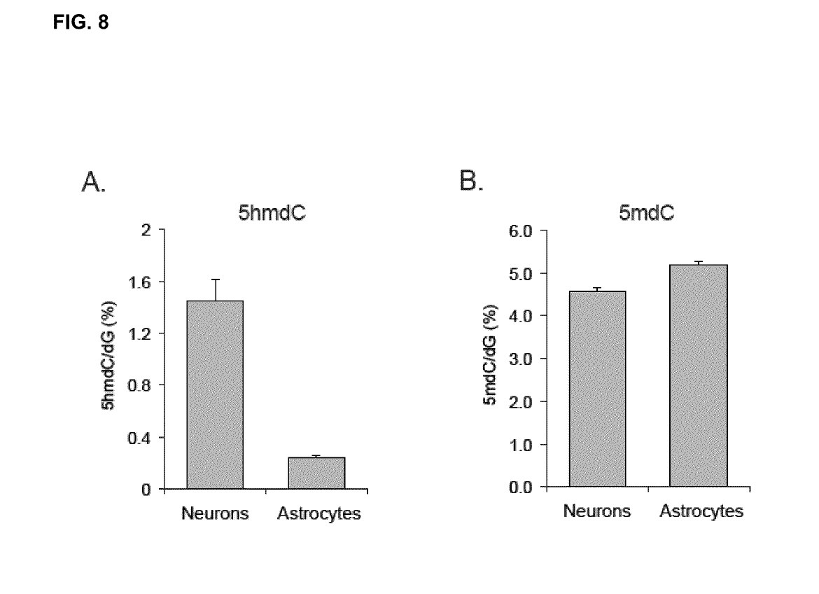

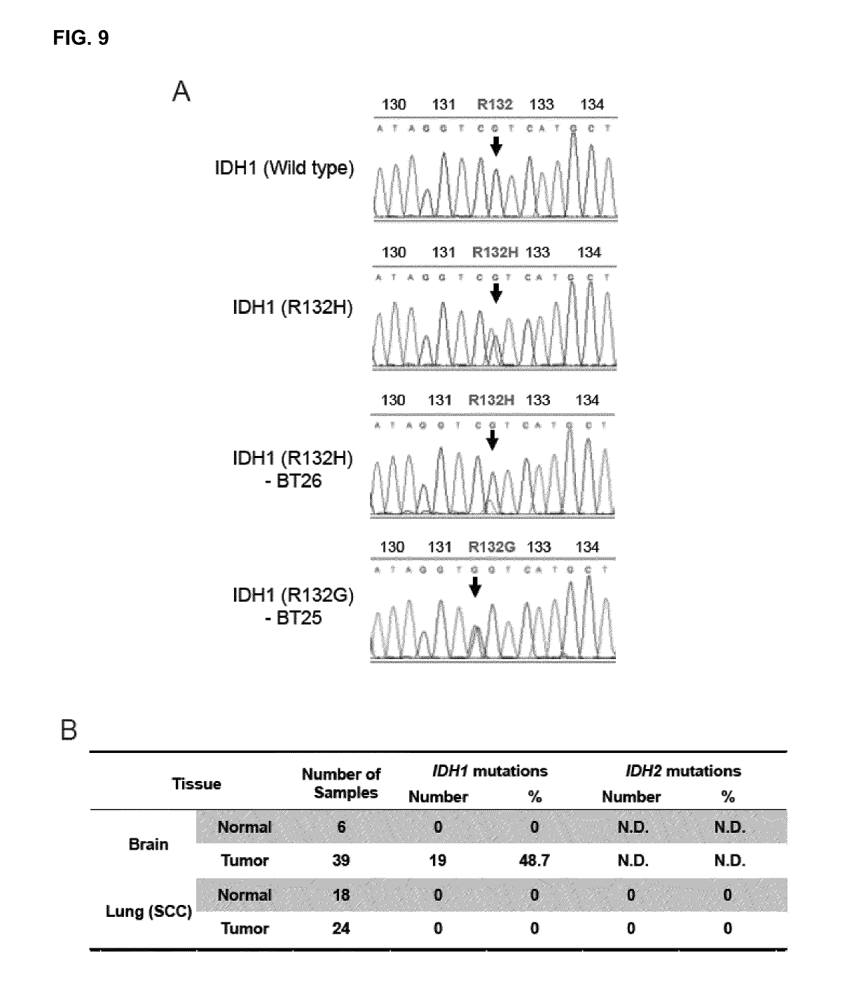

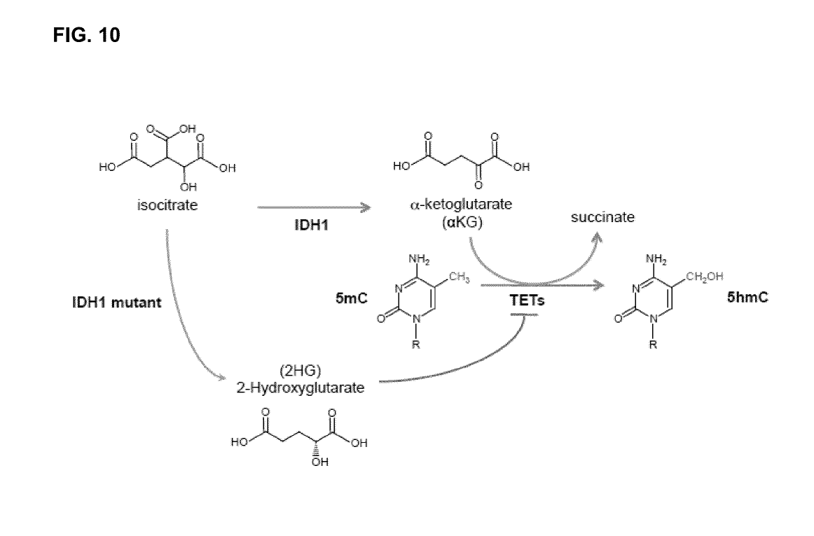

Next, the 2 modified 2′-deoxynucleosides were analyzed in 6 normal brain DNA samples and in 33 stage II and III astrocytomas (astrocytic gliomas) and in 2 glioblastomas. High levels of 5hmdC were observed in normal human brain prefrontal cortex DNA (FIG. 7A), in which 5hmdC was between 0.82% and 1.18% of dG. 5hmdC and 5mdC levels were also measured in astrocytes and in neurons from human fetal brain. Levels of 5hmdC were higher (1.45% 5hmdC/dG) in neurons than in astrocytes (0.23% 5hmdC/dG; FIG. 8). In brain tumors, levels of 5hmdC were significantly lower relative to normal brain (FIG. 7A). Some astrocytomas contained only 0.03% to 0.04% of 5hmdC, a reduction of more than 30-fold (P=1.55×10⁻¹¹; unpaired t test). Because astrocytomas initiate in neural stem cells or glial progenitor cells, their decreased level of 5hmdC may be due to either the malignant state or to the cell of origin of these tumors. The varying levels of 5hmdC in tumors did not correlate with patient age or whether the tumor was stage II or III or with patient survival. Levels of 5mdC showed only a small reduction in some brain tumors (FIG. 7B; P=0.3; unpaired t test). There was no correlation between levels of 5hmdC and levels of 5mdC. A substantial portion of stages II and III gliomas contain mutations in IDH1 and much more rarely, in IDH2 (Yan et al. 2009). The mutation status of IDH1 at codon 132 was determined (FIG. 9). 16 stage II/III tumors with the typical codon R132H mutation and 17 stage II/III tumors without any IDH1 mutation were identified. The R132H IDH1 mutation produces a neomorphic enzyme with the capacity to generate 2HG (Dang et al. 2009). IDH1-mutant tumors were expected to have lower levels of 5hmdC according to the presumed role of 2HG as an inhibitor of TET oxidases (see FIG. 10). Unexpectedly, however, the levels of 5hmdC were evenly distributed between the low and high ranges, both in IDH1 wild type and in IDH1-mutant tumors (FIG. 7A; P=0.53; t test, nonpaired). This finding is in contrast to a previous report, which observed a significant reduction of 5hmC in IDH1-mutant gliomas by immunohistochemistry (Xu et al. 2011b). Similarly, IDH1-mutant and wild-type cases did not show differences in levels of 5mdC (FIG. 7B).

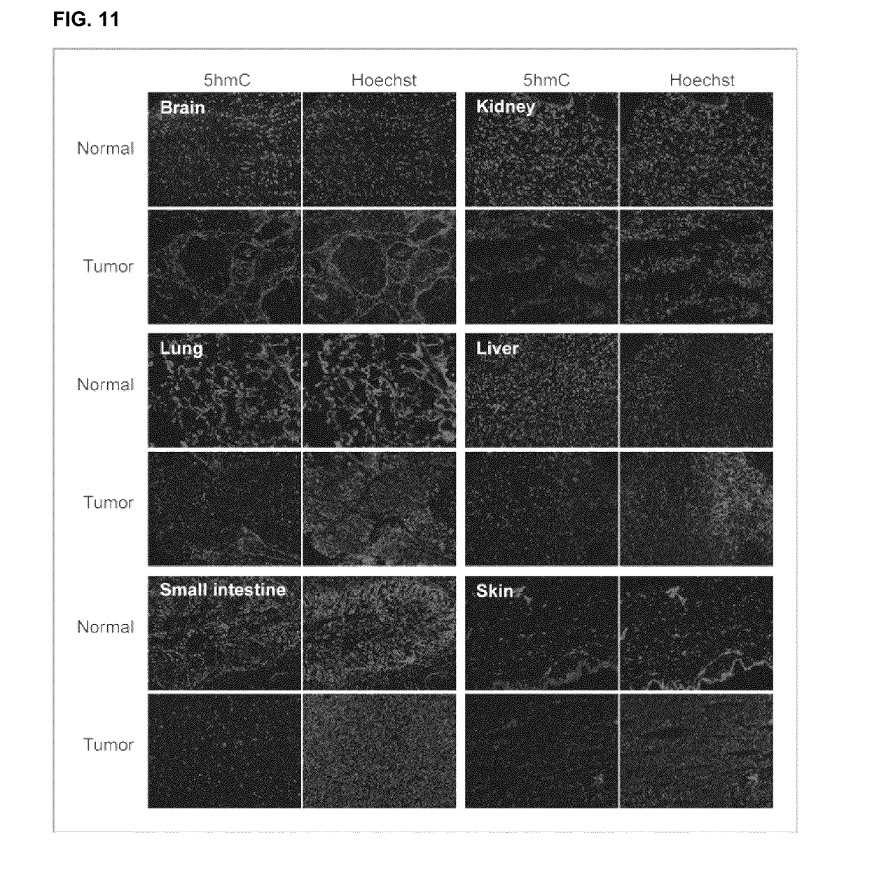

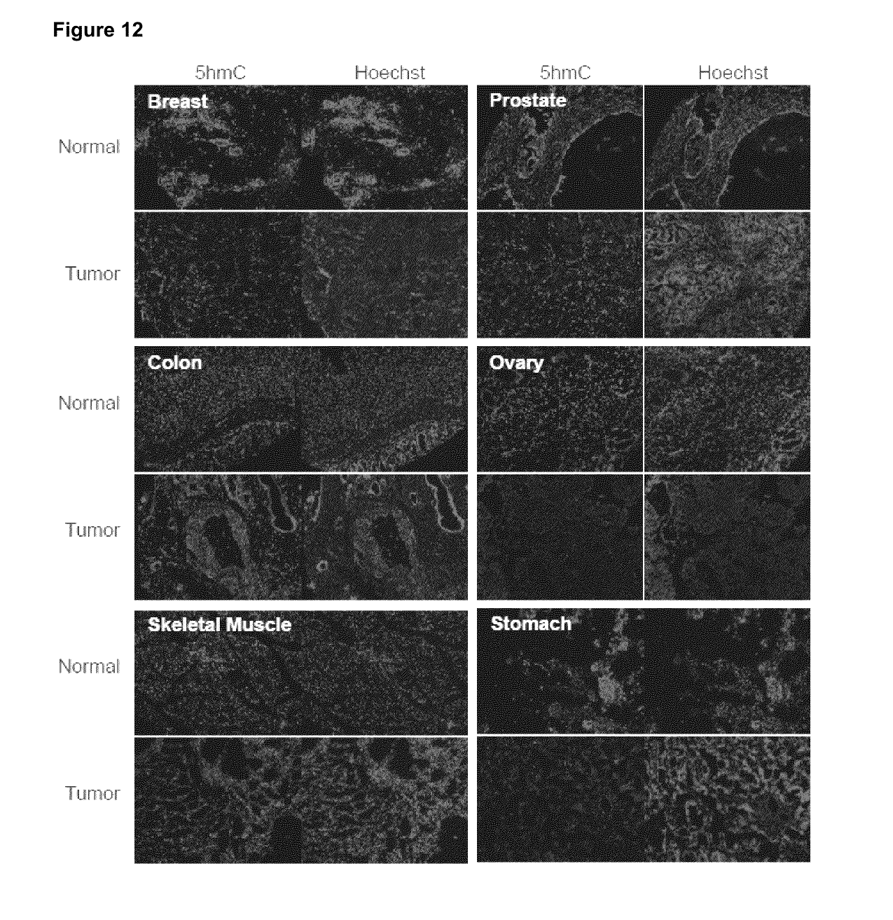

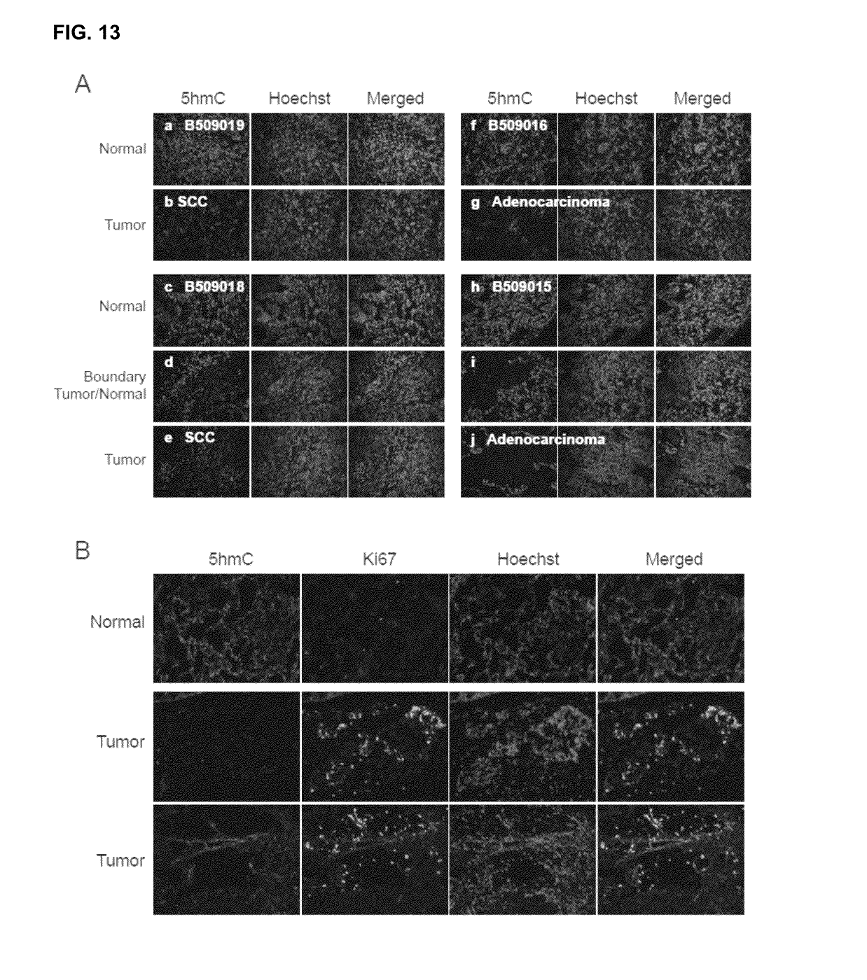

To investigate whether loss of 5hmdC is a feature of human cancers in general, immunohistochemical staining was conducted with an anti-5hmC antibody (FIGS. 11-12). This antibody was previously verified and used for detecting 5hmC in early embryos (Jin et al. 2011; Iqbal et al. 2011). Normal tissue sections and corresponding tumor sections were stained with this antibody. Substantial 5hmC staining was observed in almost all cells in most normal tissues, however, staining in corresponding tumors was universally decreased with only a few cells (<10%) staining positive for 5hmC. The only exception was a tumor originating in the colon (FIG. 12). Additional lung tumor slides were also analyzed (FIG. 13) including tumor and adjacent normal lung (FIG. 13A).

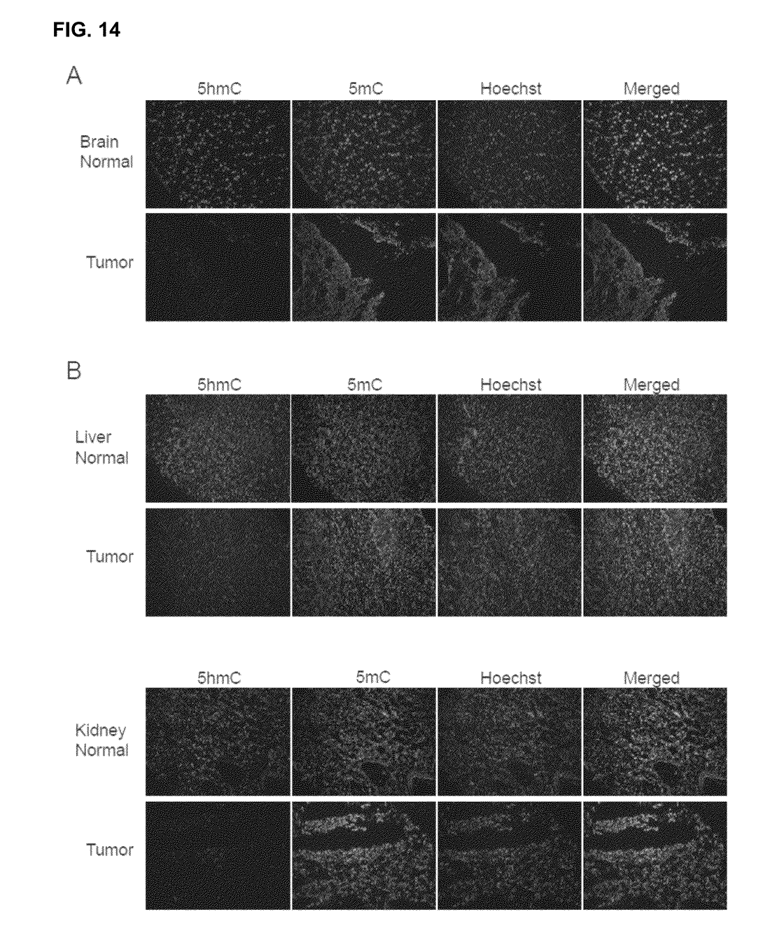

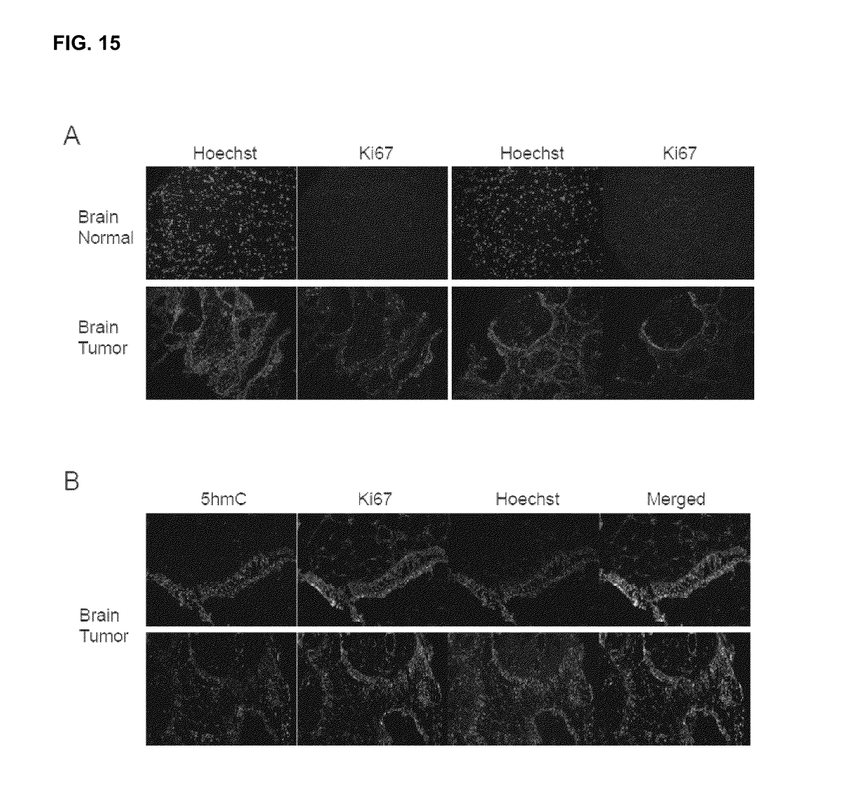

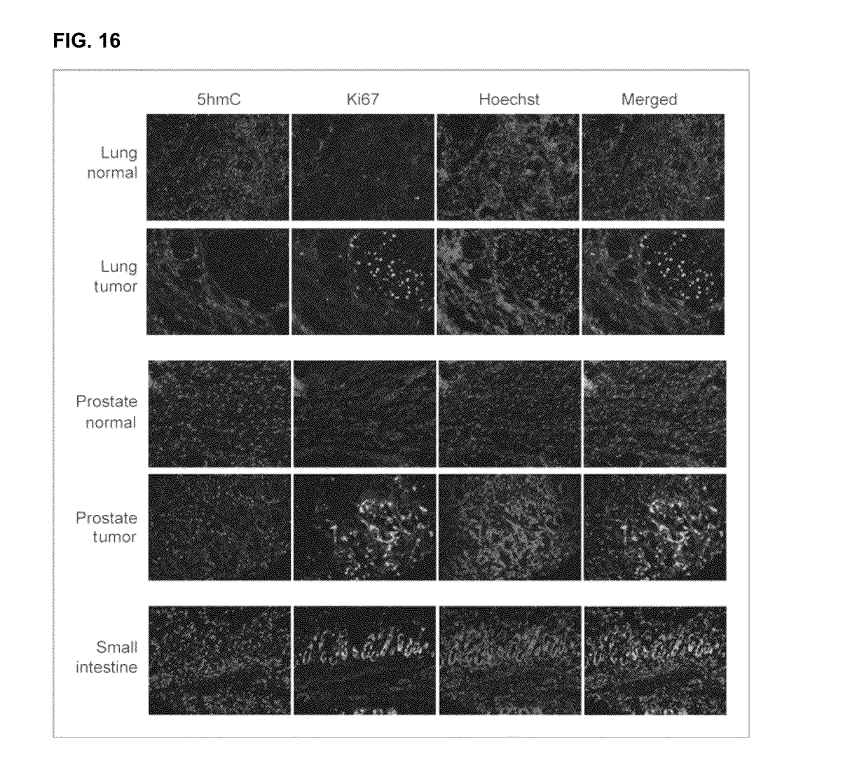

In contrast, substantial decrease of 5mC was not observed in the tumors when parallel staining of several normal and tumor sections for 5mC was performed using an anti-5mC antibody (FIG. 14). This indicates that the loss of 5hmdC is not simply due to loss of 5mdC in tumors. It was then determined whether reduced staining of 5hmdC in tumors is due to increased cell proliferation by using an anti-Ki67 antibody to stain proliferating cells. Sections of normal brain were almost devoid of Ki67 antigen but brain tumors contained many Ki67-positive cells that lacked 5hmC staining (FIG. 15). Similarly, there was little Ki67 staining in normal lung, but in adjacent carcinoma tissue, mutually exclusive staining of Ki67 and 5hmC was observed (FIGS. 13 and 16).

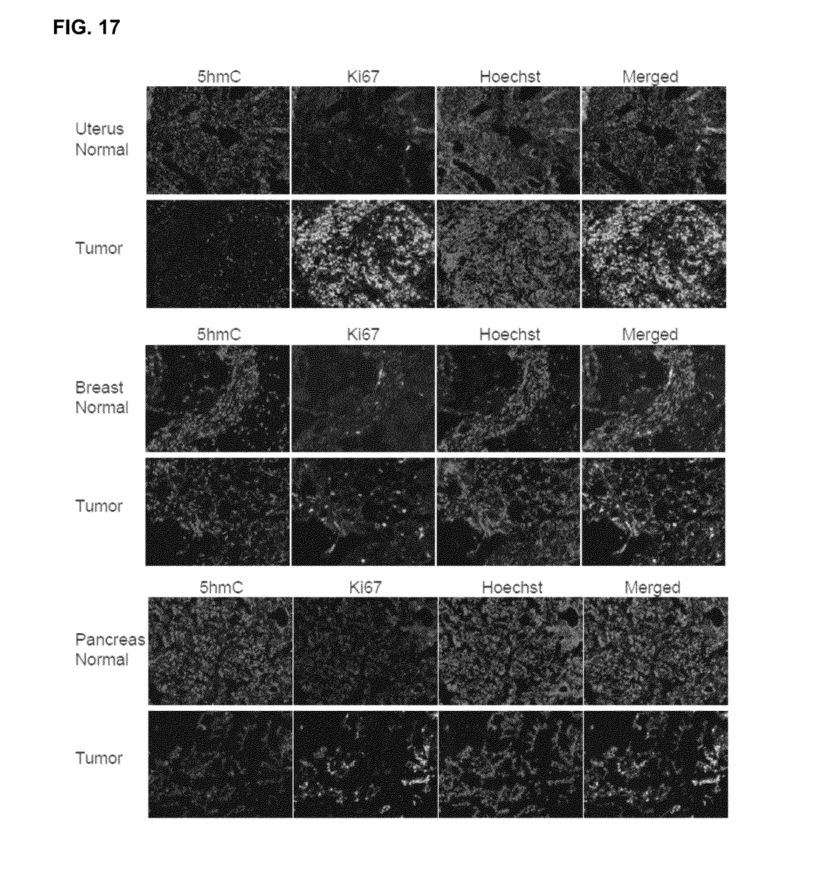

The same staining pattern was also observed in sections of breast, pancreatic tumors, and uterus tumors (FIG. 17). Tissue sections of normal small intestine showed strong Ki67 staining for proliferating cells at the bottom of crypts with lack of 5hmC staining, whereas the more differentiated cells contained high levels of 5hmC and lacked Ki67 staining (FIG. 16). Thus, one explanation for the loss of 5hmdC in tumors is the enhanced rate of cell proliferation in tumors that could lead to a passive loss of 5hmdC, which is not a substrate for DNA methyltransferase 1 (DNMT1). DNMT1 copies DNA methylation patterns shortly after DNA replication acting on sequences that contain 5mC on only the parental DNA strand. However, DNMT1 cannot methylate a substrate that contains 5hmC in place of 5mC (Valinluck & Sowers 2007). Although not all cells that lack 5hmC staining in the tumors are Ki67 positive, this may be due to a previous proliferative stage, leading to permanent loss of 5hmC.

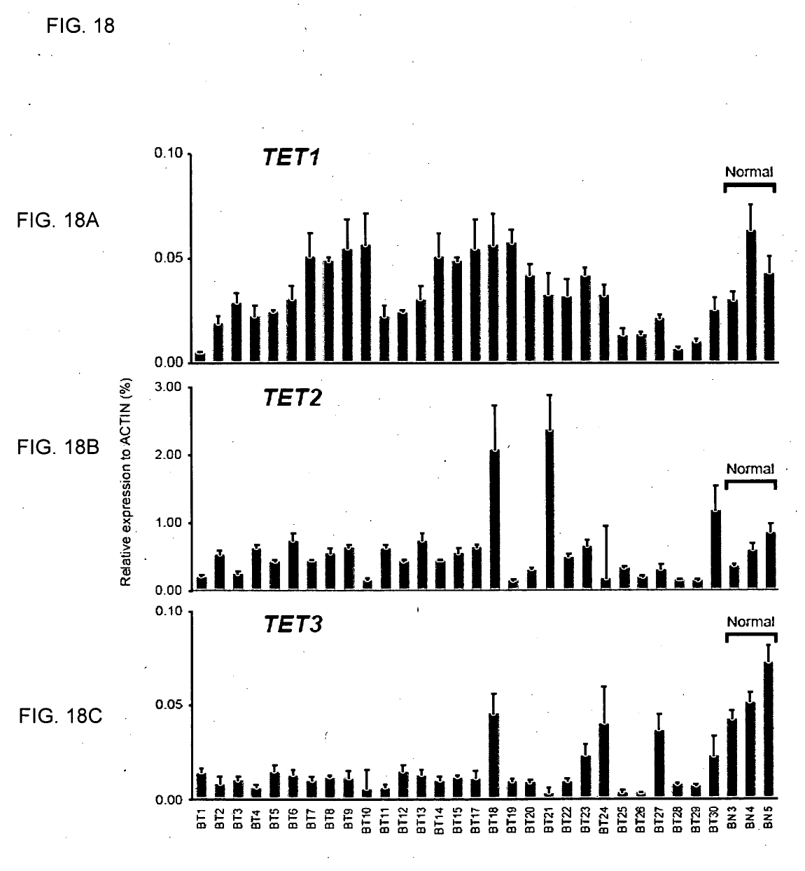

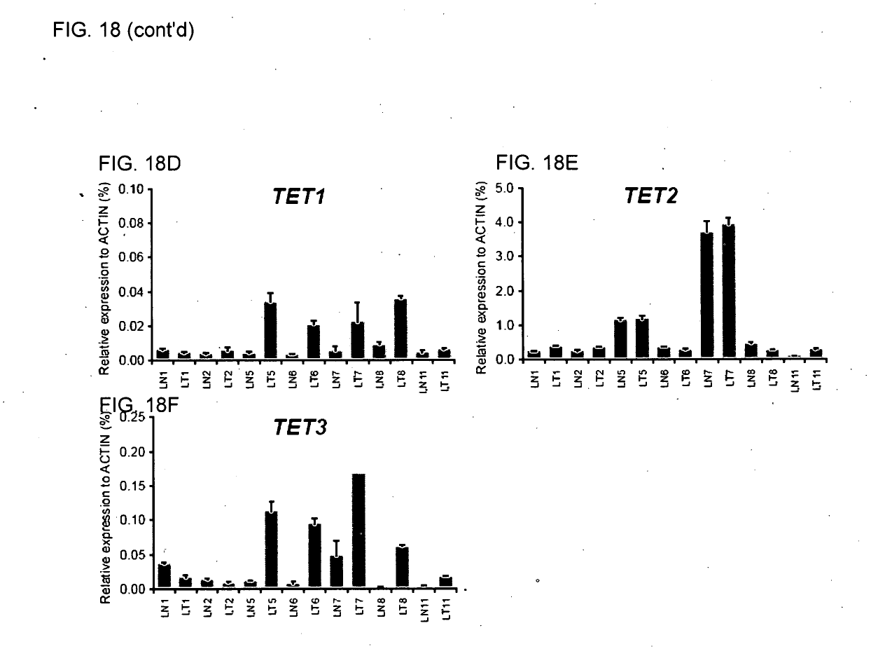

The inverse relationship between 5hmC levels and cell proliferation may also reflect intrinsic differences in cell type, as reflected by the comparison between differentiated cells and undifferentiated cancer cells that carry stem cell-like properties or originate from somatic stem cells. Another alternative is the possible existence of aberrations in 5hmC production or elimination pathways in tumors. Mutations in TET genes have not been reported in solid tumors. There was no substantial reduction of TET gene expression in lung and brain tumors relative to normal tissue as confirmed by reverse transcriptase PCR (FIG. 18A-B). The loss of 5hmdC in tumors may have profound effects on DNA methylation patterns. For example, if 5hmC is an intermediate in DNA demethylation, its loss at specific genomic locations may make these sequences more prone to acquire methylation.

It will be important to understand the mechanisms of how 5hmC is lost in tumors. Finally, loss of 5hmC may become a useful molecular biomarker for cancer detection and diagnosis, optionally in conjunction with Ki67 staining.

### REFERENCES

The references, patents and published patent applications listed below, and all references cited in the specification above are hereby incorporated by reference in their entirety, as if fully set forth herein.
Cao H, Wang Y. Collisionally activated dissociation of protonated 20-deoxycytidine, 20-deoxyuridine, and their oxidatively damaged derivatives. J Am Soc Mass Spectrom 2006; 17:1335-41.
Dang L, White D W, Gross S, Bennett B D, Bittinger M A, Driggers E M, et al. Cancer-associated IDH1 mutations produce 2-hydroxyglutarate. Nature 2009; 462:739-44.
Delhommeau F, Dupont S, Della Valle V, James C, Trannoy S, Masse A, et al. Mutation in TET2 in myeloid cancers. N Engl J Med 2009; 360: 2289-301.
Ficz G, Branco M R, Seisenberger S, Santos F, Krueger F, Hore T A, et al. Dynamic regulation of 5-hydroxymethylcytosine in mouse ES cells and during differentiation. Nature 2011; 473:398-402.
Figueroa M E, Abdel-Wahab O, Lu C, Ward P S, Patel J, Shih A, et al. Leukemic IDH1 and IDH2 mutations result in a hypermethylation phenotype, disrupt TET2 function, and impair hematopoietic differentiation. Cancer Cell 2010; 18:553-67.
Globisch D, Munzel M, Muller M, Michalakis S, Wagner M, Koch S, et al. Tissue distribution of 5-hydroxymethylcytosine and search for active demethylation intermediates. PLoS One 2010; 5:e15367.
Iqbal K, Jin S G, Pfeifer G P, Szabo P E. Reprogramming of the paternal genome upon fertilization involves genome-wide oxidation of 5-methylcytosine. Proc Natl Acad Sci USA 2011; 108:3642-7.
Ito S, D'Alessio A C, Taranova O V, Hong K, Sowers L C, Zhang Y. Role of Tet proteins in 5mC to 5hmC conversion, ES-cell self-renewal and inner cell mass specification. Nature 2010; 466:1129-33.
Jin S G, Wu X, Li A X, Pfeifer G P. Genomic mapping of 5-hydroxymethyl-cytosine in the human brain. Nucleic Acids Res 2011; 39:5015-24.
Ko M, Huang Y, Jankowska A M, Pape U J, Tahiliani M, Bandukwala H S, et al. Impaired hydroxylation of 5-methylcytosine in myeloid cancers with mutant TET2. Nature 2010; 468:839-43.
Koh K P, Yabuuchi A, Rao S, Huang Y, Cunniff K, Nardone J, et al. Tet1 and Tet2 regulate 5-hydroxymethylcytosine production and cell lineage specification in mouse embryonic stem cells. Cell Stem Cell 2011; 8:200-13.
Kriaucionis S, Heintz N. The nuclear DNA base 5-hydroxymethylcytosine is present in Purkinje neurons and the brain. Science 2009; 324:929-30.
LaFrancois C J, Fujimoto J, Sowers L C. Synthesis and characterization of isotopically enriched pyrimidine deoxynucleoside oxidation damage products. Chem Res Toxicol 1998; 11:75-83.
Noushmehr H, Weisenberger D J, Diefes K, Phillips H S, Pujara K, Berman B P, et al. Identification of a CpG island methylator phenotype that defines a distinct subgroup of glioma. Cancer Cell 2010; 17:510-22.
Pastor W A, Pape U J, Huang Y, Henderson H R, Lister R, Ko M, et al. Genome-wide mapping of 5-hydroxymethylcytosine in embryonic stem cells. Nature 2011; 473:394-7.
Song C X, Szulwach K E, Fu Y, Dai Q, Yi C, Li X, et al. Selective chemical labeling reveals the genome-wide distribution of 5-hydroxymethylcy-tosine. Nat Biotechnol 2011; 29:68-72.
Szwagierczak A, Bultmann S, Schmidt C S, Spada F, Leonhardt H. Sensitive enzymatic quantification of 5-hydroxymethylcytosine in genomic DNA. Nucleic Acids Res 2010; 38:e181.
Tahiliani M, Koh K P, Shen Y, Pastor W A, Bandukwala H, Brudno Y, et al. Conversion of 5-methylcytosine to 5-hydroxymethylcytosine in mammalian DNA by MLL partner TET1. Science 2009; 324: 930-5.
Valinluck V, Sowers L C. Endogenous cytosine damage products alter the site selectivity of human DNA maintenance methyltransferase DNMT1. Cancer Res 2007; 67: 946-50.
Williams K, Christensen J, Pedersen M T, Johansen J V, Cloos P A, Rappsilber J, et al. TET1 and hydroxymethylcytosine in transcription and DNA methylation fidelity. Nature 2011; 473:343-8.
Wu S C, Zhang Y. Active DNA demethylation: many roads lead to Rome. Nat. Rev Mol Cell Biol 2010; 11:607-20.
Wu H, D'Alessio A C, Ito S, Wang Z, Cui K, Zhao K, et al. Genome-wide analysis of 5-hydroxymethylcytosine distribution reveals its dual function in transcriptional regulation in mouse embryonic stem cells. Genes Dev 2011; 25:679-84.
Xu Y, Wu F, Tan L, Kong L, Xiong L, Deng J, et al. Genome-wide regulation of 5hmC, 5mC, and gene expression by Tet1 hydroxylase in mouse embryonic stem cells. Mol Cell 2011a; 42:451-64.
Xu W, Yang H, Liu Y, Yang Y, Wang P, Kim S H, et al. Oncometabolite 2-hydroxyglutarate is a competitive inhibitor of alpha-ketoglutarate-dependent dioxygenases. Cancer Cell 2011b; 19:17-30.
Yan H, Parsons D W, Jin G, McLendon R, Rasheed B A, Yuan W, et al. IDH1 and IDH2 mutations in gliomas. N Engl J Med 2009; 360:765-73.

What is claimed is:

1. A method for detecting or diagnosing cancer in a subject comprising:
measuring a test level of 5hmC in a biological sample from the subject; and
determining that the subject has a malignant cancer when the test level of 5hmC is lower than that of a control level of 5hmC.

2. The method of claim 1, further comprising measuring a test level of Ki67 in the biological sample and determining that the subject has a malignant cancer when the test level of Ki67 is higher than that of a control level of Ki67.

3. The method of claim 1, wherein the cancer is bone cancer, bladder cancer, brain cancer, breast cancer, cancer of the urinary tract, carcinoma, adenocarcinoma, cervical cancer, colon cancer, esophageal cancer, gastric cancer, head and neck cancer, hepatocellular cancer, liver cancer, lung cancer, lymphoma and leukemia, malignant mesenchymoma, melanoma, neuroblastoma, ovarian cancer, pancreatic cancer, pituitary cancer, prostate cancer, rectal cancer, renal cancer, rhabdomyosarcoma, sarcoma, testicular cancer, thyroid cancer, or uterine cancer.

4. The method of claim 1, wherein the biological sample is a tumor tissue sample.

5. The method of claim 4, wherein the test level of 5hmC is measured using immunohistochemistry (IHC).

6. The method of claim 4, wherein the biological sample is a DNA sample isolated from the tumor tissue.

7. The method of claim 6, wherein the test level of 5hmC is measured using liquid chromatography/tandem mass spectrometry (LC/MS-MS) or anti-5hmC antibody-based methods to detect 5hmC, for example immuno dot blot or ELISA.

8. The method of claim 6, wherein the test level of 5hmC is measured as 5hmdC.

9. The method of claim 1, wherein the control level of 5hmC is measured using a normal adjacent tissue from the same subject.

10. The method of claim 1, wherein the control level of 5hmC is measured using a normal sample from a second subject or a population of normal subjects.

11. A method for detecting or diagnosing cancer in a subject comprising:
measuring a test level of 5hmdC in a tumor tissue sample from the subject, wherein the test level of 5hmdC is measured using LC/MS-MS; and
determining that the subject has a malignant cancer when the test level of 5hmC is lower than that of a control level of 5hmC.

12. A method for detecting or diagnosing cancer in a subject comprising:
measuring a test level of 5hmc in a biological sample from the subject;
measuring a test level of Ki67 in the biological sample; and
determining that the subject has a malignant cancer when (1) the test level of 5hmc is lower than that of a control level of 5hmc and (2) the test level of Ki67 is higher than that of a control level of Ki67.

13. The method of claim 11, wherein the cancer is bone cancer, bladder cancer, brain cancer, breast cancer, cancer of the urinary tract, carcinoma, adenocarcinoma, cervical cancer, colon cancer, esophageal cancer, gastric cancer, head and neck cancer, hepatocellular cancer, liver cancer, lung cancer, lymphoma and leukemia, malignant mesenchymoma, melanoma, neuroblastoma, ovarian cancer, pancreatic cancer, pituitary cancer, prostate cancer, rectal cancer, renal cancer, rhabdomyosarcoma, sarcoma, testicular cancer, thyroid cancer, or uterine cancer.

14. The method of claim 11, wherein the biological sample is a tumor tissue sample.

15. The method of claim 13, wherein the test level of 5hmC, the test level of Ki67, or both is measured by IHC.

16. The method of claim 13, wherein biological sample is a DNA sample isolated from the tumor tissue.

17. The method of claim 15, wherein the test level of 5hmC is measured using LC/MS-MS or anti-5hmC antibody-based methods.

18. The method of claim 15, wherein the test level of 5hmC is measured as 5hmdC.

19. The method of claim 11, wherein the control level of 5hmC is measured using a normal adjacent tissue from the same subject.

20. The method of claim 11, wherein the control level of 5hmC is measured using a normal sample from a second subject or a population of normal subjects.


SEQUENCE LISTING (4 sequences)

SEQ ID NO: 1 - DNA, 20 bp, Artificial Sequence
  Synthetic primer
tgccaccaacgaccaagtca

SEQ ID NO: 2 - DNA, 22 bp, Artificial Sequence
  Synthetic primer
catgcaaaatcacatatttgcc

SEQ ID NO: 3 - DNA, 20 bp, Artificial Sequence
  Synthetic primer
tgaaagatggcggctgcagt

SEQ ID NO: 4 - DNA, 20 bp, Artificial Sequence
  Synthetic primer
ggggtgaagaccattttgaa